(19) United States
(12) Patent Application Publication
(10) Pub. No.: US 2012/0189693 A1
(43) Pub. Date: Jul. 26, 2012

(54) ORAL DOSAGE FORMS

(75) Inventors: Christopher C. Dick, New Hope, PA (US); David F. Erkoboni, Pennington, NJ (US); Charanjit R. Behl, Hauppauge, NY (US); Gary Bubb, Lebanon, NJ (US)

(73) Assignee: Elite Laboratories, Inc., Northvale, NJ (US)

(21) Appl. No.: 13/379,486

(22) PCT Filed: Jun. 25, 2010
(86) PCT No.: PCT/US10/39961

§ 371 Date: Mar. 21, 2012

Related U.S. Application Data
(60) Provisional application No. 61/220,320, filed on Jun. 25, 2009.

(51) Int. Cl. A61K 9/20; A61K 9/36; A61K 31/485; A61P 25/04; A61P 29/00; A61P 33/10; A61P 9/06; A61P 31/04; A61P 31/12; A61P 7/02; A61P 25/24; A61P 3/10; A61P 25/08; A61P 31/10; A61P 19/06; A61P 9/12; A61P 25/06; A61P 35/00; A61P 15/10; A61P 37/06; A61P 33/02; A61P 5/14; A61P 25/22; A61P 25/20; A61P 25/18; A61P 9/04; A61P 25/16; A61P 1/00; A61P 9/10; A61P 21/02; A61P 19/10; A61P 3/04; A61P 25/28; A61K 9/28; A61K 9/42; A61K 9/38; A61K 9/34; B29C 59/02; B29B 9/00; A61K 9/32

(52) U.S. Cl. 424/465; 424/464; 424/482; 424/480; 514/282; 424/474; 424/476; 424/477; 424/481; 264/118; 264/109

(57) ABSTRACT

Aspects of the present invention are directed to oral dosage forms comprising a compressed microtablet, wherein said microtablet has a major dimension that is between about 0.25 mm and about 1.0 mm and comprises at least about 0.01 weight percent of at least one pharmaceutically active agent that is distributed substantially throughout said microtablet. Additional aspects of the present invention are directed to methods for producing compressed microtablets having a major dimension that is between about 0.25 mm and about 1.0 mm.

### CROSS REFERENCE TO RELATED APPLICATIONS

This application claims the benefit of priority to U.S. Provisional Patent Application Ser. No. 61/220,320, filed Jun. 25, 2009, which is herein incorporated by reference in its entirety.

### TECHNICAL FIELD

The present invention relates, inter alia, to oral dosage forms comprising pharmaceutically active agents and, in particular, to compressed microtablets and oral dosage forms comprising them.

### BACKGROUND

Small particles containing pharmaceutically active agents are well known in the art and are often incorporated into capsules or tablets. These particles provide for the ability to, for example, combine multiple active agents into a single dosage form. Additionally, the use of multiple particles of different types in a single dosage form provides one way to adjust the different release profile of the active agent.

Typically, these small particles have an inert core coated with the active agent. Traditional inert cores such as sugar non-pareils, microcrystalline cellulose beads, and wax beads used as substrates to deliver pharmaceutically active agents have several drawbacks, including the potential for rupture of the coating membrane due to swelling or osmotic forces resulting in rapid release or “dumping” of the active agent contained in the pellet and an increased particle size of the pellet dosage form due to the presence of the inert core.

Compressed tablets provide an alternative to drug coated inert cores and offer several benefits. For example, compressed tablets may have a smoother outer surface than traditional inert cores, which could be important if the tablet is coated with a non-releasing or controlled release membrane because a smoother surface could result in a more uniform membrane coating and a more consistent release of the active agent. Additionally, compressed tablets can have a more uniform size distribution which can result in lower intra- and inter-batch tablet size variability, which should result in a more consistent coating and therefore a more predictable and consistent release profile.

Although compressed tablets have several advantages over inert cores, their use has been limited due to the inability to make these tablets in a size small enough for certain pharmaceutical applications.

### SUMMARY

One aspect of the present invention relates to oral dosage forms comprising compressed microtablets, wherein the microtablet has a major dimension that is between about 0.25 and about 1.0 mm and comprises at least about 0.01 weight percent of at least one pharmaceutically active agent that is distributed substantially throughout the microtablet.

Additional aspects of the present invention are directed to methods comprising the steps of compressing a composition comprising at least one pharmaceutically active agent into a microtablet having a major diameter of between about 0.25 mm and about 1.0 mm.

Further embodiments of the invention relate to methods comprising the steps of compressing a composition comprising at least one pharmaceutically active agent into a microtablet array having a height of between about 0.25 mm and about 1 mm and a width of between about 1 mm and about 4 mm; breaking the compressed microtablet array perpendicular to its width to form multiple microtablets; and, optionally, polishing the components to form at least two relatively spherical compressed microtablets having a major dimension that is between about 0.25 and about 1.0 mm.

### BRIEF DESCRIPTION OF THE DRAWINGS

The foregoing and other aspects of the present invention will become apparent from the following detailed description of the invention when considered in conjunction with the accompanying drawings. For the purpose of illustrating the invention, there is shown in the drawings embodiments that are presently preferred, it being understood, however, that the invention is not limited to the specific instrumentalities disclosed. In the drawings:

FIG. 1 is a side view of a compressed microtablet tooling assembly;

FIG. 2A is a view of a compressed microtablet tooling assembly during compression of microtablets;

FIG. 2B is a view of a compressed microtablet tooling assembly before or after compression in open mode;

FIG. 3A is a side view of an upper body of a compressed microtablet tooling assembly;

FIG. 3B is a top view of an upper body of a compressed microtablet tooling assembly;

FIG. 4A is a top view of an upper pin holder of a compressed microtablet tooling assembly;

FIG. 4B is a side perspective view of an upper pin holder of a compressed microtablet tooling assembly;

FIG. 4C is a bottom perspective view of an upper pin holder of a compressed microtablet tooling assembly;

FIG. 5A is a side view of an upper pin of a compressed microtablet tooling assembly;

FIG. 5B is a side perspective view of an upper pin of a compressed microtablet assembly;

FIG. 6A is a side view of a die of a compressed microtablet tooling assembly;

FIG. 6B is a top perspective view of a die of a compressed microtablet tooling assembly;

FIG. 6C is a bottom perspective view of a die of a compressed microtablet tooling assembly;

FIG. 7 is a side view of a pressed microtablet tooling assembly;

FIG. 8 is a side view of an upper body or lower body of a compressed microtablet tooling assembly;

FIG. 9 is a top view of the compression surface of an upper body or lower body of a compressed microtablet tooling assembly;

FIG. 10 is a cross-sectional view of the microtablet array formed between the compressed microtablet tooling upper body and lower body; and

FIG. 11 is a cross-sectional view of a microtablet array prior to separation.

### DETAILED DESCRIPTION OF ILLUSTRATIVE EMBODIMENTS

One aspect of the present invention provides oral dosage forms, i.e., dosage forms that are designed to be administered via the oral cavity of a patient to deliver one or more pharmaceutically active agents. A wide variety of pharmaceutically active agents can be used in the present invention. Indeed, virtually any agent that can be produced in a form that is sufficiently free flowing to uniformly fill a die and has particles small enough to fit within a die, such as, for example, a particle size of about 10-400 microns can be utilized in the present invention.

Suitable active ingredients agents include, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppresants, anti-protozoal agents, anti-thyroid agents, anxiolytic agents, sedatives, hypnotics, neuroleptics, beta-blockers, cardiac inotropic agents, corticosteroids, diuretics, anti-parkinsonian agents, gastrointestinal agents, histamine receptor antagonists, keratolytics, lipid regulating agents, anti-anginal agents, COX-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, hematinics, vitamins, minerals, nutrients, cosmeceuticals, diagnostic agents, or a nutritional agents. and mixtures thereof.

In one embodiment, the active agent is hydrophobic. Hydrophobic active agents are compounds with little or no water solubility. Intrinsic water solubilities (i.e., water solubility of the unionized form) for hydrophobic active ingredients are less than about 1% by weight, and typically less than about 0.1% or 0.01% by weight. In a particular aspect of this embodiment, the active ingredient is a hydrophobic drug. In other particular aspects the hydrophobic active ingredient is a hydrophobic nutrient, cosmeceutical, diagnostic agent or nutriceutical.

Representative hydrophobic active ingredients include, for example, acitretin, acyclovir, albendazole, aldactone, alprazolam, amiloride, amiodarone, amlodipine, amphotericin B, anagrelide hydrochloride, atazanavir, atorvastatin, atovaquone, azathioprine sodium, azithromycin, beclomethasone, benzonatate, bicalutamide, budesonide, busulfan, cabergoline, calcitriol, candesartan, carbamezepine, carotenes, carvedilol, cefuroxime axetil, celecoxib, cholecalciferol, cilostazol, ciprofloxacin, cisapride, clarithromycin, clemastine fumarate, clofibrate, clopidogrel, coenzyme Q10, colestipol hydrochloride, cyclosporin, danazol, dantrolene, dapsone, desloratadine, diclofenac, dicoumarol, diflunisal, digoxin, dehydroepiandrosterone, dihydrotachysterol, dipyridamole, dirithromycin, efavirenz, enalaprilat, eprosartan, ergocalciferol, essential fatty acid sources, esomeprazole, etodolac, famotidine, felodipine, fenofibrate, finasteride, fluconazole, flurbiprofen, fosphenytoin, furazolidone, gemfibrozil, glipizide, glimepiride, glucagon, glutethimide, griseofulvin, ibuprofen, irbesartan, irinotecan, isotretinoin, itraconazole, ivermectin, ketoconazole, ketoprofen, lamotrigine, lansoprazole, leflunomide, loperamide, loratadine, lovastatin, L-thyroxine, lycopene, meclizine hydrochloride, medroxyprogesterone, mifepristone, mefloquine, megestrol acetate, methoxsalen, methyclothiazide, metronidazole, metyrosine, miconazole, minoxidil, mycophenolate mofetil, nabumetone, nadolol, naproxen, nelfinavir, nifedipine, nisoldipine, nilutamide, nitrofurantoin, olmesartan, omeprazole, oestradiol, oxaprozin, paricalcitol, pioglitazone, prednisone, prednisolone, probenecid, progesterone, repaglinide, rescinnamine, rifabutin, rifapentine, rimexolone, ritonavir, rofecoxib, rosiglitazone, saquinavir, sertraline, sibutramine, sildenafil citrate, simvastatin, sirolimus, spironolactone, sucralfate, sulfasalazine, sumatriptan, tacrolimus, tadalafil, tamoxifen, tamsulosin, targretin, telmisartan, terbinafine, tetrahydrocannabinol, topiramate, topotecan, toremifene, tretinoin, triamterene, ubidecarenone, ursodiol, valsartan, vardenafil, venlafaxine, verteporfin, vigabatrin, vitamin A, vitamin D, vitamin E, vitamin K, zafirlukast, zileuton, and zopiclone. Furthermore, salts, isomers, and derivatives of the above-listed hydrophobic active ingredients may also be used, as well as mixtures thereof.

In another embodiment, the active ingredient is a hydrophilic compound. Apparent water solubilities for hydrophilic active ingredients are greater than about 1.0% by weight, and typically greater than about 5 or 10% by weight. In a particular aspect of this embodiment, the hydrophilic active ingredient is a hydrophilic drug. In other particular aspects, the hydrophilic active ingredient is a cosmeceutical, a diagnostic agent, or a nutritional agent.

Representative hydrophilic active ingredients include, for example, acarbose, acyclovir sodium, acetazolamide hydrochloride, alendronate sodium, amantadine hydrochloride, ambenonium chloride, aminocaproic acid, amitriptyline hydrochloride, amphetamine, aztreonam, bacitracin, balsalazide disodium, belladonna, benazepril hydrochloride, benzphetamine hydrochloride, bethanechol chloride, biperiden hydrochloride, bleomycin sulfate, bupropion hrdrochloride, butabarbital sodium, captopril, carbidopa, carbinoxamine maleate, capecitabine, cefuroxime sodium, cephalexin, citalopram hydrobromide, chlorpheniramine maleate, chloroquine, cetirizine hydrochloride, citalopram hydrobromide, chlordiazepoxide hydrochloride, cetirizine hydrochloride, clidinium bromide, clindamycin and clindamycin derivatives, clodronate, clomipramine hydrochloride, clonidine hydrochloride, clopidogrel bisulfate, clorazepate dipotassium, cloxacillin sodium, codeine sulfate, cosyntropin, cromolyn sodium, cyclobenzaprine hydrochloride, cysteamine, desferrioxamine, desipramine hydrochloride, desmopressin, diatrizoate meglumine and diatrizoate sodium, dicloxacillin sodium, dicyclomine hydrochloride, didanosine, diltiazem hydrochloride, dimenydrinate, diphenhydramine hydrochloride, donepezil hydrochloride, doxycycline hyclate, etidronate disodium, erythromycin, etidronate disodium, famciclovir, fentanyl citrate, flecainide acetate, fluoxetine, fluvastatin sodium, fosphenytoin sodium, frovatriptan sodium, ganciclovir, galantamine hydrochloride, glycopyrrolate, granisetron hydrochloride, ipodate sodium, hydralazine hydrochloride, hydromorphone hydrochloride, imipramine hydrochloride, ketorolac trimethamine, lamivudine, leucovorin calcium, Levetiracetam, levodopa, levofloxacin, levorphanol tartrate, lincomycin hydrochloride, lisinopril, losartan potassium, loracarbef, mecamylamine hydrochloride, meperidine hydrochloride, mepenzolate bromide, mesalamine, methadone hydrochloride, methenamine hippurate, methotrexate sodium, methscopolamine, methyldopa, methylphenidate, metformin hydrochloride, metoprolol, midazolam hydrochloride, miglitol, minocycline hydrochloride, misoprostol, mitoxantrone, montelukast sodium, morphine sulfate, nalbuphine hydrochloride, naltrexone, neurontin, nizatidine, ofloxacin, olsalazine sodium, oxybutynin, oxyphenonium bromide, pantoprazole sodium, paroxetine hydrochloride, pentoxifylline, phenoxybenzamine hydrochloride, phenylalanine, phenytoin sodium, pralidoxime chloride, pravastatin sodium, pregabalin, propafenone, propantheline bromide, propranolol hydrochloride, pseudoephedrine hydrochloride, pyridostigmine bromide, quinapril hydrochloride, rabeprazole sodium, risedronate sodium, ribavirin, rimantadine hydrochloride, salmeterol xinafoate, sitagliptin phosphate, sotalol, stavudine, sulfoxone sodium, tacrine hydrochloride, terazosin, terbutaline sulfate, timolol maleate, tramadol hydrochloride, triprolidine hydrochloride, urea, vancomycin, valacyclovir hydrochloride, valproic acid, valsartan, varenicline tartrate, verapamil hydrochloride, vitamin B12, warfarin sodium, zalcitabine, zidovudine, zolpidem tartrate, pharmaceutically acceptable salts, isomers and derivatives thereof, and mixtures thereof.

In a further embodiment, the pharmaceutically active agent may be an opioid agonist or antagonist. Representative opioids agonists include, for example, hydrocodone, morphine, hydromorphone, oxycodone, codeine, levorphanol, meperidine, methadone, oxymorphone, buprenorphine, fetanyl and derivatives thereof, dipipanone, heroin, tramadol, etorphine, dihyroetorphine, butorphanol, levorphanol, and mixtures thereof. In other embodiments, the pharmaceutically active agent may be an opioid antagonist. Representative opioid antagonists include naltrexone, naloxone, nalmephene, cyclazacine, levallorphan, and mixtures thereof.

Suitable amounts of pharmaceutically active agents may vary depending upon the agent, the disease or disorder sought to be treated, and/or the approximate body weight of the patient. For example, amounts of naltrexone suitable to block the euphoric effects of 40 mg of oxycodone typically are from about 0.04 mg to about 100 mg, preferably from about 2 mg to about 60 mg and most preferably 4 mg to 30 mg. Comparable ratios (e.g., from 0.001-1 to 2.5-1 naltrexone to oxycodone) can be used regardless of the dose of oxycodone.

It will be understood that a dosage form of the invention can itself include constituent dosage forms. Thus, for example, the present invention embraces dosage forms in which microtablets of pharmaceutically active agents are contained within a gelatin capsule, compressed tablet, or suspension.

The compressed microtablets of the present invention have a size that makes them feasible for use in pharmaceutical applications, such as in a capsule or tablet form. For example, the compressed microtablets may have a major dimension of between about 0.25 mm and about 1.0 mm. In a preferred embodiment, the compressed microtablets have a major dimension of between about 0.4 mm and about 0.9 mm. In another preferred embodiment, the compressed microtablets have a major dimension of between about 0.5 mm and about 0.8 mm. As used herein, the range of between about 0.25 mm and about 1.0 mm is inclusive. For example, the recited range should be construed as including ranges “0.25 to 0.9”, “0.25 to 0.8”, “0.3 to 0.7”, and the like. The compressed microtablets may have an aspect ratio (i.e., a ratio of major/longest dimension to minor/shortest dimension) of between about 1:0.5 and about 1:4, preferably between about 1:0.9 and 1:1.1. Additionally, the compressed microtablets may have a variety of shapes. Preferably, the shape of the microtablet is substantially spherical. Microtablets according to the invention comprise from about 0.01 to about 99.0 weight percent of at least one pharmaceutically active agent, preferably from about 5 to about 75 weight percent, and most preferably from about 10 to about 50 weight percent.

In accordance with the present invention, the at least one pharmaceutically active agent preferably is dispersed substantially throughout the microtablet, i.e., dispersed such that the mean volume within the core that does not contain pharmaceutically active agent is not greater than about 0.01 cc active agent. The microtablets of the invention thus are to be contrasted with prior dosage forms in which the pharmaceutically active agent (even if present in an equivalent absolute amount) is disposed in a more localized (i.e. less dispersed) manner.

The pressed microtablets may bear a membrane. The membrane may comprise one or more polymers. The membrane can result in immediate release or some form of controlled release of the active pharmaceutical ingredient. Controlled release forms can include delayed, enteric, extended or sustained release. The membrane may comprise one or more water soluble polymers. Preferably, the water soluble polymer is physiologivcally acceptable. The membrane may comprise one or more water-retardant polymers. Preferably, the water-retardant polymer is physiologically acceptable, and substantially prevents or controls the release of the pharmaceutically active agent. In addition, the water retardant polymer may optionally be water insoluble. Representative classes of polymers include cellulose polymer, acrylic polymer, acrylic acid copolymers, methacrylic acid polymers, methacrylic acid copolymers, shellac, zein, or hydrogenated vegetable oil or waxes.

Suitable cellulose polymers include ethylcellulose, cellulose acetate, cellulose propionate (lower, medium, and higher molecular weight), cellulose acetate propionate, cellulose acetate butyrate, cellulose acetate phthalate, cellulose triacetate, cellulose ether, cellulose ester, cellulose, ester ether, cellulose, cellulose acrylate, cellulose diacylate, cellulose, triacylate, cellulose acetate, cellulose diacetate, cellulose triacetate, mono, di, and tricellulose alkanylates, mono, di, and tricellulose aroylates, mono, di, and tricellulose alkenylates, cellulose trivalerate, cellulose trilaurate, cellulose tripatmitate, cellulose trisuccinate, cellulose trioctanoate, cellulose disuccinate, cellulose dipalmitate, cellulose dioctanoate, cellulose dipentanoate, coesters of cellulose such as cellulose acetate butyrate, and cellulose acetate octanoate butyrate. Additional cellulose polymers include acetaldehyde dimethyl cellulose acetate, cellulose acetate ethylcarbamate, cellulose acetate methylcarbanate, and cellulose acetate dimethylaminocellulose acetate. Aqueous dispersions of some of the polymers are available, such as Aquacoat, ethyl cellulose dispersion (FMC Corp.), Aquacoate CPD cellulose acetate dispersion (FMC Corp.), Surelease ethylcellulose dispersion (Colorcon), Surteric, hydroxypropyl methylcellulose phthalate (Colorcon), and AQOAT hypromellose acetate succinate (Shin-Etsu) and can also be used in certain embodiments. Preferred cellulose polumers include cellulose esters such as ethyl cellulose and cellulose, ethers such as hypromellose and pH sensitive cellulose acetate phthalate and succinate salts. In certain embodiments, suitable polymers include polylactic acid, polyglycolic acid, or a co-polymer of the polylactic and polyglycolic acid.

In certain embodiments, the water retardant polymer may be an acrylic polymer. Suitable acrylic polymers include acrylic acid and methacrylic acid copolymers, methyl methacrylate copolymers, ethoxyethyl methacrylate, cyanoethyl methacrylate, poly(acrylic acid), poly(methacrylic acid), methacrylic acid alkylamide copolymer, poly(methyl methacrylate), polymethacrylate, poly (methyl methacrylate) copolymer, polyacrylamide, aminoalkyl methacrylate copolymer, poly(methacrylic acid anhydride), and glycidyl methacrylate copolymers. Preferred acrylic polymers include copolymers of methylmethacrylate and ethylacrylate and copolymers of acrylate and methacrylates. Aqueous dispersions of such polymers are commercially available as Eudragit RS 30D, Eudragit RL 30D, Eudragit NE 30D, Eudragit NE 40D, and Eudragit NM 30D from Evonik Rohm GmbH, Darmstadt, Germany. Particularly preferred are non-ionic polyv(ethylacrylate-co-methylmethacrylate) polymers in which the molar proportions of the ethyl acrylate and methyl methacrylate monomer units, respectively, are in the ratio of about 2:1 and/or that have average molecular weights of about 800,000 Daltons (such as Eudragit NE 30D, Eudragit NE 40D, and Eudragit NM 30D). Further examples of suitable acrylic polymers include ammonio methacrylate copolymer Types A and B, methacrylic acid copolymer, Type A and B, and/or polymers of methacrylic acid and methacrylates (e.g., available as powders as Eudragit S and Eudragit L).

In certain embodiments, the oral dosage form may comprise between about 2 and about 800 weight percent increase in weight after application of the, preferably between about 10 and about 500 weight percent increase, and more preferably between about 20 and about 400 weight percent increase. The weight increase results in a coated microtablet composition comprising between about 2 and about 89 weight percent of coating membrane, preferably, between about 9 and about 83 weight percent of coating membrane, and more preferably between about 16 weight percent and about 80 weight percent of coating membrane.

The membrane may be disposed directly upon the core or upon an intervening layer or structure. The membrane can be applied by any of the techniques known in the art. Typically, the core is coated with a solution of water-retardant polymer and the solvent is allowed to evaporate.

The membrane may optionally comprise a lubricant/anti-tacking agent such as, for example, calcium stearate, magnesium stearate, zinc stearate, stearic acid, glyceryl monostearate, talc or a combination thereof. In one preferred embodiment, with a preferred opioid antagonist, naltrexone, the membrane contains magnesium stearate admixed with the water-retardant polymer, preferably Eudragit NE-30D—ethyl acrylate and methyl methacrylate copolymer dispersion. The lubricant may function to prevent agglomeration of the compressed microtablets during processing and may also help to prevent or control release of the pharmaceutically active agent from the oral dosage form. In some embodiments, the membrane contains an amount of magnesium stearate, or other lubricant, sufficient to provide non-release of the pharmaceutically active agent for up to about 36 hours after administration of the dosage form to a human being. In other embodiments, the membrane contains an amount of calcium stearate, or other lubricant, sufficient to result in a controlled and extended release of the pharmaceutically active agent for up to about 36 hours. Preferably, the dried membrane contains between about 0.5 and about 200 weight percent increase lubricant/anti-tacking agent(s), more preferably between about 1 and about 100 weight percent increase, and most preferably between about 5 and about 50 weight percent increase.

In practice, compressed microtablets will include not only the pharmaceutically active agent but also at least one excipient. Suitable excipients include compression aids, binder agents, glidants, disintegrants, lubricants, or a combination thereof, among others. Suitable compression aids include microcrystalline cellulose, lactose, dicalcium phosphate, sucrose, stearic acid, polyethylene glycol, waxes such as microcrystalline wax, carnuba wax and the like or a combination thereof, among others. Suitable binder agents include, for example, hypromellose, hydroxyethyl cellulose, hydroxypropyl cellulose, methyl cellulose, ethyl cellulose, cellulose acetate phthalate, hypromellose acetate phthalate, polyvinyl acetate phthalate polyvinyl pyrrolidone, polyvinyl alcohol, copovidone—copolymer of vinylpyrrolidone and vinyl acetate, a carbomer, amino methylacrylate copolymer, methacrylic acid copolymers, acrylic polymers, aqueous dispersions of methacrylates and ethylcellulose, and the like. Suitable binder agents include those included in the opioid antagonist layer and are described in detail herein. Suitable glidants include talc, silicon dioxide, metallic silicates, or a combination thereof, among others. Suitable disintegrants include starch, croscarmellose sodium, crospovidone, sodium sarch glycolate or a combination thereof, among others. Suitable lubricants include calcium strearate, magnesium stearate, zinc stearate, stearic acid, talc, hydrogenated vegetable oil, glyceryl monostearate, or a combination thereof, among others. In addition the cores can bear suitable coating materials. In certain embodiments, the excipient(s) constitute between about 1.00 and about 99.99 weight percent of the oral dosage form. In a preferred embodiment, the excipient(s) may constitute between about 5 and about 80 weight percent of the uncoated compressed microtablet and between 10 and about 60 weight percent in a more preferred embodiment. For example, the oral dosage form may comprise between about 5 and about 99.99 weight percent compression aid, between about 0.5 and about 50.0 weight percent binding agent, between about 0.1 and about 20.0 weight percent glidant, between about 0 and about 10.0 weight percent of disintegrant, and between about 0.1 and about 10.0 weight percent lubricant in the uncoated compressed microtablet.

The compressed microtablet and/or membrane thereon may be coated with an optional sealing layer. For example, the sealing layer may include a water soluble polymer such as hypromellose (preferably 3 to 6 cps, more preferably 6 cps), hydroxyethyl cellulose, hydroxypropyl cellulose, methyl cellulose, polyvinylpyrrolidone, polyvinyl alcohol and the like. Preferably, hypromellose (6 cps) and polyvinyl alcohol, and most preferably, polyvinyl alcohol is employed in the sealing layer. In addition, the sealing layer may optionally contain a lubricant, such as for example, calcium stearate, magnesium stearate, zinc stearate, stearic acid, talc or a combination thereof. The optional sealing layer coated between the opioid antagonist and the membrane may comprise between about 0.5 and about 450 weight percent of the oral dosage form.

The membrane may also be coated with an enteric layer comprising an enteric coating polymer. Enteric polymers can include but are not limited to cellulose, acrylic and vinyl based polymers. Suitable enteric polymer coatings include, for example, cellulose acetate phthalate from solvent or as aqueous dispersion, Aquacoat CAP (FMC Corp.); methacrylic acid copolymer frm solvent or dispersion, for example Eudragit L30D-55 (Evonik Industries), methacrylic acid copolymer dispersion Type A and B, for example, Eudragit L-100 and S-100 (Evonik Industries), hydroxymethylcellulose phthalate, Polyvinyl Acetate Phthalate from solvent or as aqueous dispersion. Sureteric (Colorcon), or any combination thereof. The enteric layer may further comprise a plasticizer. Preferably, the enteric coating polymer is Eudragit L 30D. Suitable plasticizers include, for example, triethyl citrate, polyethylene glycol, dibutyl phthalate, diethylphthalate and triacetin. The enteric layer, which is pH dependant and resistant to gastric fluids, preferably comprises between about 0.5 and about 40 weight percent of the oral dosage form. In other embodiments of the invention, the enteric layer may also be coated with a sealing layer the same or similar to the previously described sealing layers.

The microtablet comprising a pharmaceutically active agent and/or the membrane may further comprise diluents, carriers, fillers and other pharmaceutical additives which may or may not affect the rate of release of the pharmaceutically active agent from the oral dosage form of the invention. For example, the membrane may comprise a lubricant or plasticizer. The microtablet and/or the membrane may also further contain pharmaceutically acceptable excipients such as binders, fillers, disintegrants, anti-adherents, lubricants and pharmaceutically acceptable pigments such as titanium dioxide, iron oxide and various color pigments including vegetable dyes, and the like.

To date, the formation of compressed tablets having dimensions of the microtablets of the present invention has been unattainable. There are several reasons for this difficulty. For example, there has been an inability to compress tablets with a diameter of about 1 mm or less without bending or breaking the tooling used to form the tablets. Upon the application of a force necessary to compress the material into a tablet, the tooling would bend or break. Aspects of the present invention overcome these obstacles. In one aspect of the invention, this is because tooling has been designed such that compressive force is principally applied perpendicular to the microtablet's major dimension. In another aspect of the invention, this is because the compression pins which are similar in function to the punch tips of a conventional tablet tool are less prone to distortion under pressure due to, for example, their short length and additional radial support.

Aspects of the present invention provide the ability to form microtablets having a diameter of about 1 mm or less at a rate of production that is suitable for commercial exploitation. For example, microtablets with a diameter of about 1 mm or less preferably are formed at a rate that is greater than about 5,000 microtablets per minute, or 10,000 microtablets per minute, more preferably greater than about 150,000 microtablets per minute, even more preferably greater than about 250,000 microtablets per minute.

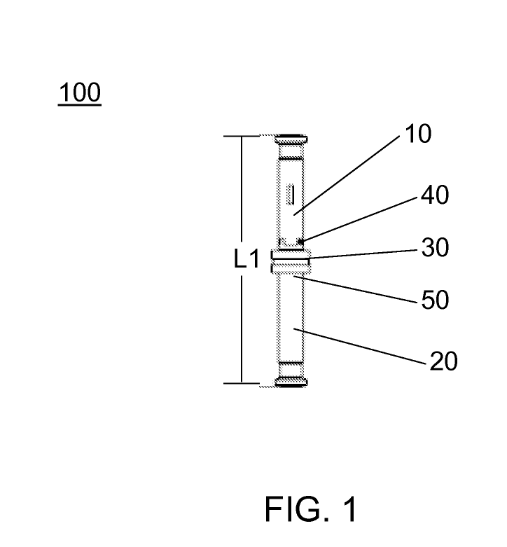

FIG. 1 shows an exemplary tooling assembly 100 for the formation of compressed microtablets having a diameter of about 1 mm or less. Assembly 100 comprises an upper body 10, a lower body 20, a die 30, an upper pin holder 40, and a lower pin holder 50. Assembly 100 is designed to work in traditional tabletting machines. For example, the overall length of assembly 100 is preferably greater than about 10.5 inches and can fit onto traditional rollers or cams. Assembly 100 may be made of any material suitable for such an assembly. Preferably assembly 100 is made of steel which is hardened after the assembly is prepared.

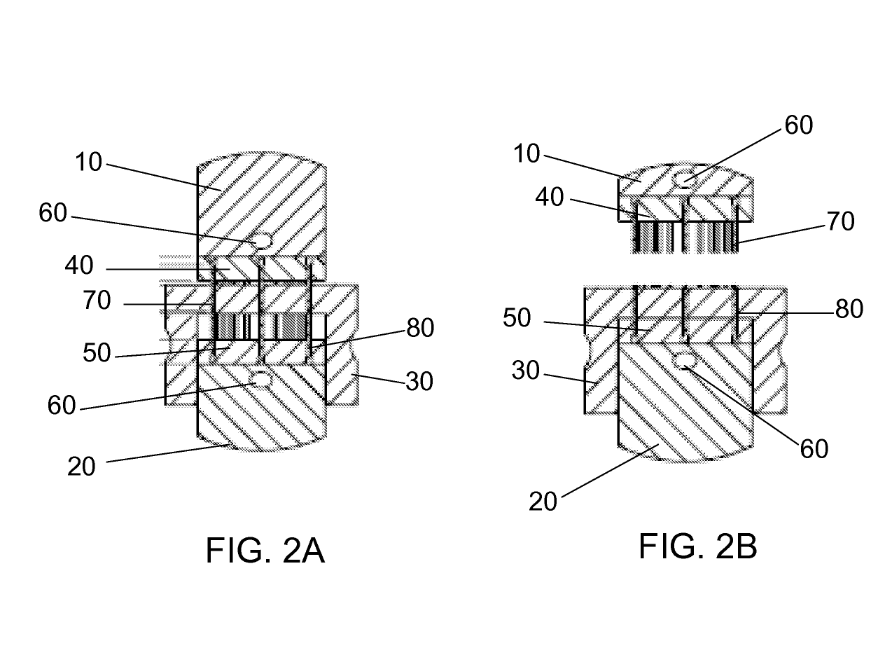

FIG. 2 shows a view of assembly 100 during (FIG. 2A) and after (FIG. 2B) compression. Upper body 10 and lower body 20 are attached to upper pin holder 40 and lower pin holder 50, respectively. The pin holders are attached to the bodies via a rod 60 that passes through the pin holders and the bodies. Further, each pin holder comprises at least one pin 70 and 80. Upon compression (FIG. 2A), upper body 10 and lower body 20 are brought together wherein the upper pins 70 and lower pins 80 contact each other to compress material into a microtablet. Upper pins 70 are pressed through die 30 and force material to consolidate against the lower pins 80. Suitable pressure for the formation of the microtablets may be between about 10 and about 500 MPa with a preferred range between about 25 and about 250 MPa and a more preferred range between about 50 and about 150 MPa. Additionally, the microtablets may be compressed to a breaking force (hardness) of between about 0.1 and about 50 Newtons, preferably between about 0.5 and about 20 Newtons. To facilitate ejection of the compressed microtablet, (FIG. 2B), lower pins 80 move upward into die 30 until the tips are flush with die 30 and upper pins 70 move upward and out of die 30. A benefit of this structure is that a significant portion, if not most, of the length of the pins 70 and 80 are supported during the compression by pin holders 40 and 50 and die 30. Such support provides for less bending/breakage of the pins during compression.

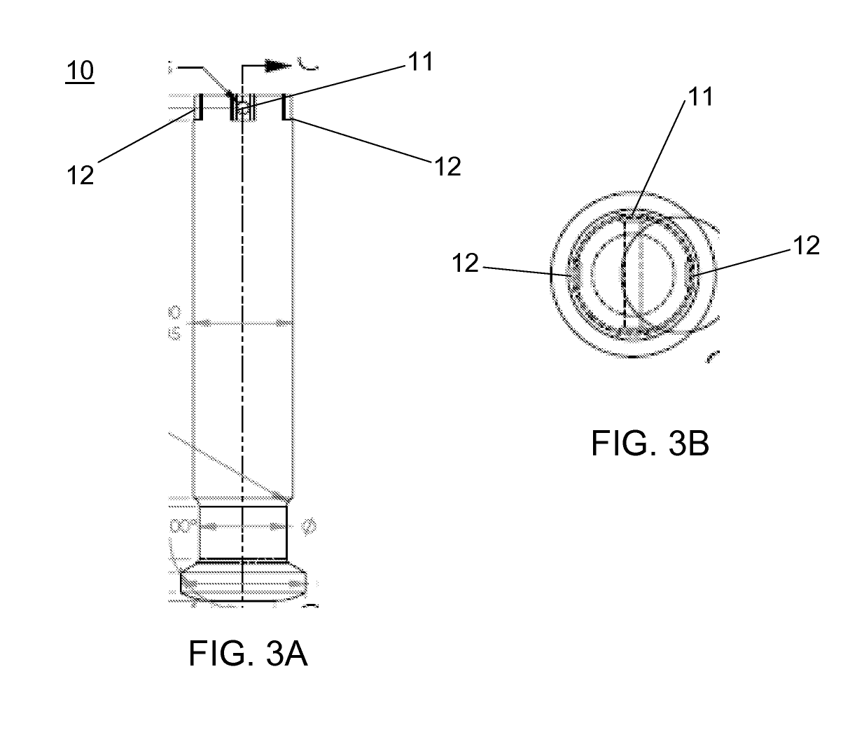

FIG. 3 shows an exemplary upper body 10. The main components of upper body 10 and lower body 20 are substantially similar. As such, both components may be in accordance with this example. Upper body 10 has a rod hole 11 through which rod 80 may be passed to attached upper pin holder 40 to upper body 10. The top of upper body 10 may also contain slots 12 into which upper pin holder 40 may fit for additional support.

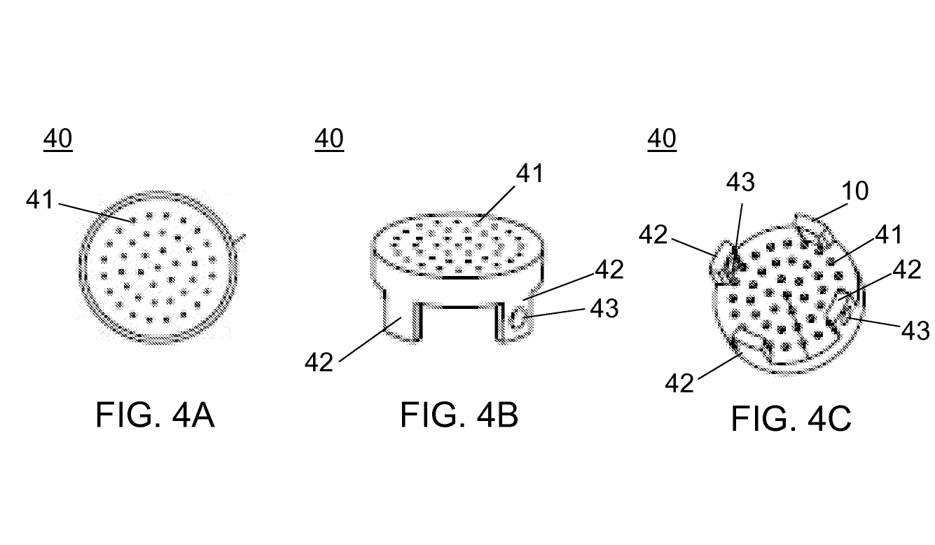

FIG. 4 shows an exemplary upper pin holder 40. The main components of upper pin holder 40 and lower pin holder 50 are substantially similar. As such, both upper pin holder 40 and lower pin holder 50 may be represented by FIG. 4. Upper pin holder 40 may comprise a number of pin holes 41 into which upper pins 70 can be placed. Preferably, pin holes 41 are tapered. In certain embodiments, pin holes 41 are tapered to an amount of 2-45 degrees. Tapering of pin holes 41 provides multiple benefits. For example, tapering provides for the release of air from the material being compressed. Such release of air reduces the risk of air bubbles in the microtablet. Additionally, tapering provides for a greater margin of error in the placement of upper pins 70 in pin holes 41 during the compression process. For example, the tapering may provide for a funneling effect of upper pins 70 into pin holes 41. The number of pin holes 41 in upper pin holder 40 may vary depending upon the needs of the user. In certain embodiments, the number of pin holes 41 may be between about 40 and about 250. In a preferred embodiment the number of pin holes 41 is 217. In another preferred embodiment, the number of pin holes 41 is 87. Upper pin holder 40 may be designed with legs 42 to fit within slots 12 of upper body 10. Additionally, at least one of leg 42, preferably two, may contain a hole 43 into which rod 80 may be placed to secure upper pin holder 40 to upper body 10.

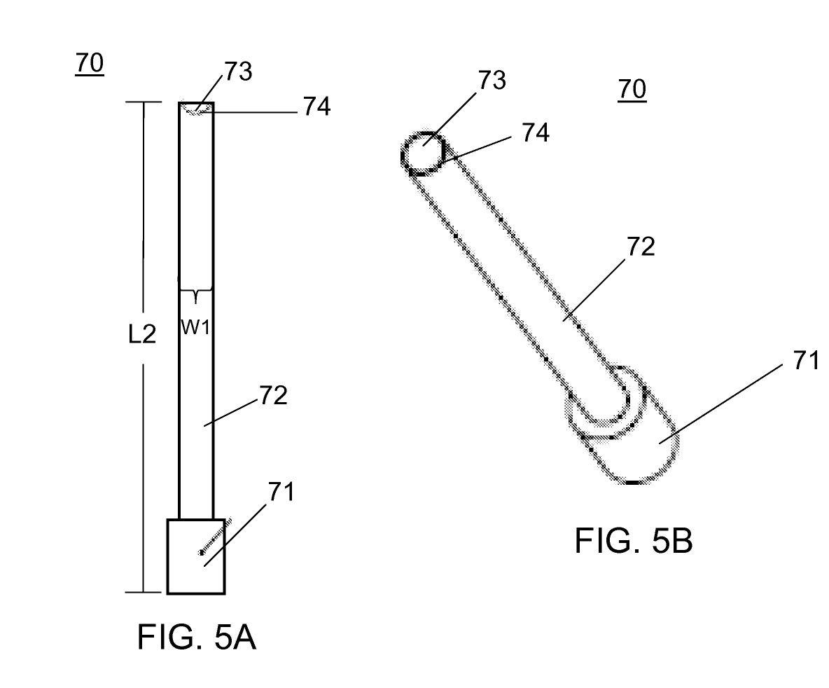

FIG. 5 shows an exemplary upper pin 70 suitable for use in the present invention. The main components of upper pin 70 and lower pin 80 are substantially similar. As such, both upper pin 70 and lower pin 80 may be represented by FIG. 5. Upper pin 70 may comprise a head portion 71 and a body portion 72. Upper pin 70 may have a length L2 of between about 1 and about 25 mm, preferably between about 2 to about 10 mm and more preferably between about 3 to about 5 mm, and a width W1 of between about 0.25 and about 1 mm. The length of upper pins 70 are substantially shorter than traditional punch tips. The decreased length of pins 70 reduces the risk of bending or breaking during compression. Upper pin 70 may comprise a concavity 73 at the tip of upper pin 70. Concavity 73 is designed to form half of the compressed microtablet upon compression of the material. Concavity 73 may be in the form of a half sphere. The diameter of the half sphere may be between about 0.25 mm and about 1 mm. Preferably, upper and lower pins 70 are removable from upper pin holder 40 and lower pin holder 50 and are replaceable. For example, if upper or lower pin 70 were to bend or break during the compression process, it could be replaced. Current devices, used for compression of tablets, do not provide for the replacement of punch tips. Thus, in current devices, if a punch tip bends or breaks, the entire unit must be replaced leading to an increase in costs.

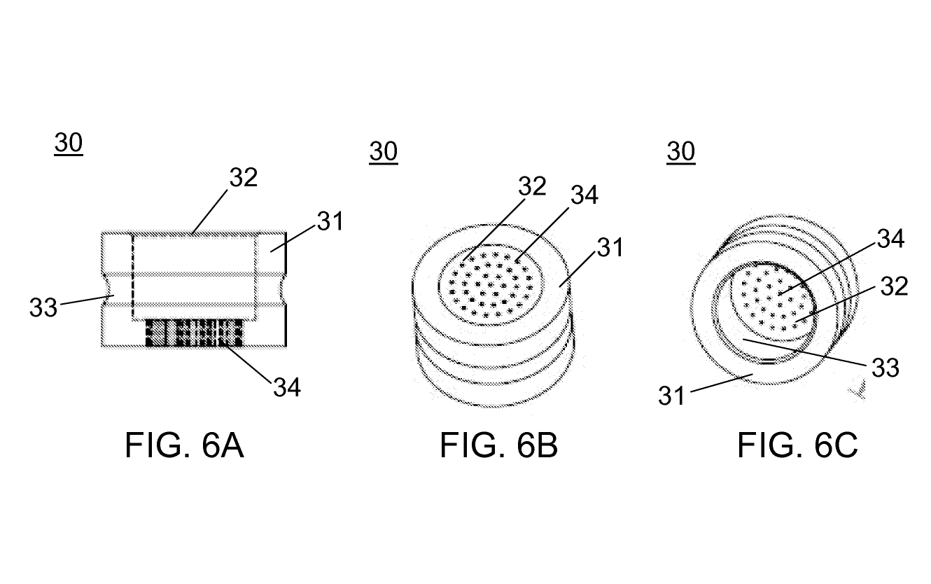

FIG. 6 shows an exemplary die 30. Die 30 comprises an exterior wall 31, a die surface 32, and an interior cavity 33. Die surface 32 contains die holes 34 corresponding to upper pins 70 and lower pins 80 attached to upper pin holder 40 and lower pin holder 50, respectively. Die holes 34 extend from die surface 32 to interior cavity 33. Die holes 34 have a length L3 of between about 2 mm and 23 mm, preferably between about 4 mm and about 10 mm. The length of die holes 34 provides for additional support of the pins during the compression process. During compression, the additional support from sidewalls of die holes 34 prevents the pins from bending or breaking since there is very little of the lower or upper pin length that is not contained within the die at the point of maximum compression. Die holes 34 are also tapered similarly to the pin holes 41 of upper pin holder 40 to provide for the release of air during compression and to act as a funnel for the pins. Additionally, the taper can act as an aid to help the flow of powder or granules into the small diameter die opening.

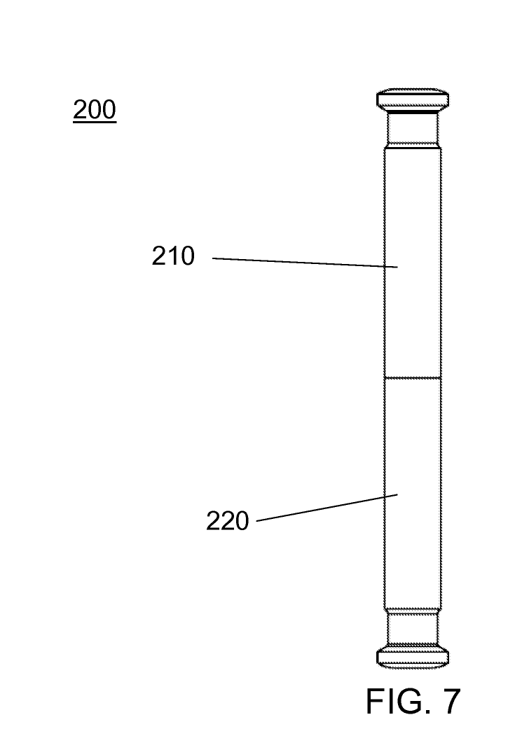

In an alternative embodiment, pressed microtablets having a diameter of about 1 mm or less can be formed by pressing a microtablet array, which can subsequently be separated into individual microtablets by breaking the array at depressed areas. There are several benefits to formation of microtablets using this process as opposed to traditional microtablet forming processes. For example, the compressive force needed to compress microtablets results in a high pressure due to the small diameter of conventional type punches. In this embodiment the force is more evenly distributed across the entire compression surface of the body and the compression pressure is reduced overall, thus preventing breakage of punch tips and other components of a traditional tablet tool. Additionally, microtablets having much smaller dimensions can be formed using this process as compared to traditional mini or micro tablet production techniques using conventional tooling. A particular embodiment for the formation of a microtablet array is shown in FIGS. 7 to 11.

FIG. 7 shows an exemplary tooling assembly 200 for the formation of compressed microtablet arrays with multiple microtablets having a diameter of about 1 mm or less. Assembly 200 comprises an upper body 210 and a lower body 220. Assembly 200 is designed to work in traditional tabletting machines. For example, the overall length of assembly 200 is preferably greater than about 10.5 inches and can fit onto traditional rollers or cams with little or no modification. Assembly 200 may be made of any material suitable for the machining of such an assembly. Preferably assembly 200 is prepared from premium steel which is later hardened.

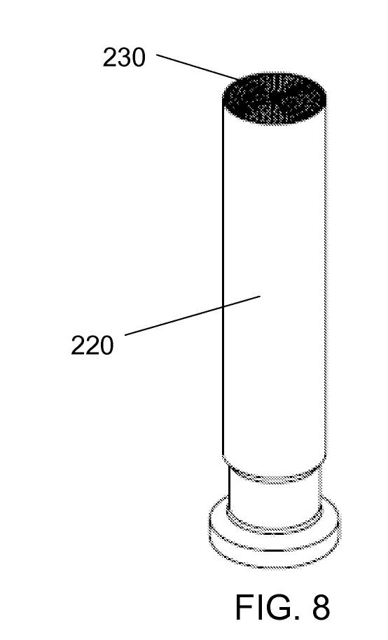

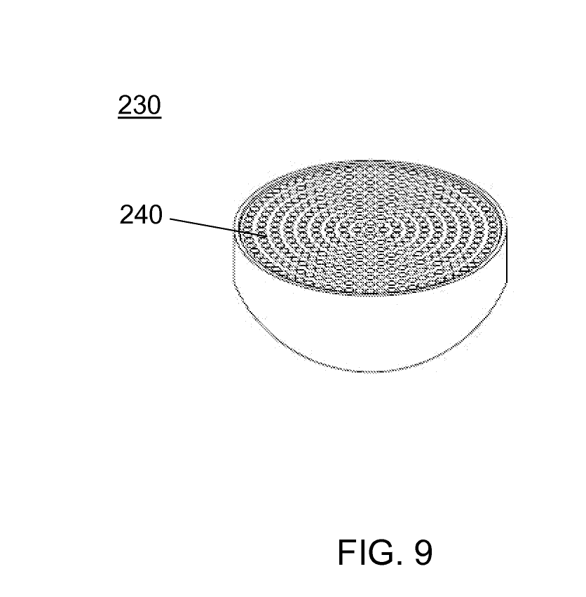

FIG. 8 shows an exemplary lower body 220. The main components of lower body 220 and upper body 210 are substantially similar. As such, both components may be in accordance with this example. Lower body 220 comprises a punch surface 230. FIG. 9 shows an exemplary punch surface 230. Punch surface 230 comprises wells 240 having a diameter of between about 0.25 mm and about 1 mm and a depth of between about 0.125 mm and about 0.5 mm. The number of wells per die surface may vary depending upon the size of the wells and the size of the punch surface. For example, punch surface 230 may have greater than about 50 wells, or 100 wells, or 200 wells. Preferably, punch surface 230 has the maximum number of wells possible for the size microtablet to be produced. This minimizes the surface area of the depressed areas resulting in increase productivity and decreased waste. Wells 240 are attached to each other by a depression 250 in punch surface 230. Depression 250 has a diameter and a depth that is less than the diameter and depth of well 240. For example, depression 250 may have a diameter of between about 0.125 mm and about 0.99 mm and a depth of between about 0.125 mm and about 0.495 mm. Depression 250 connects multiple wells 240.

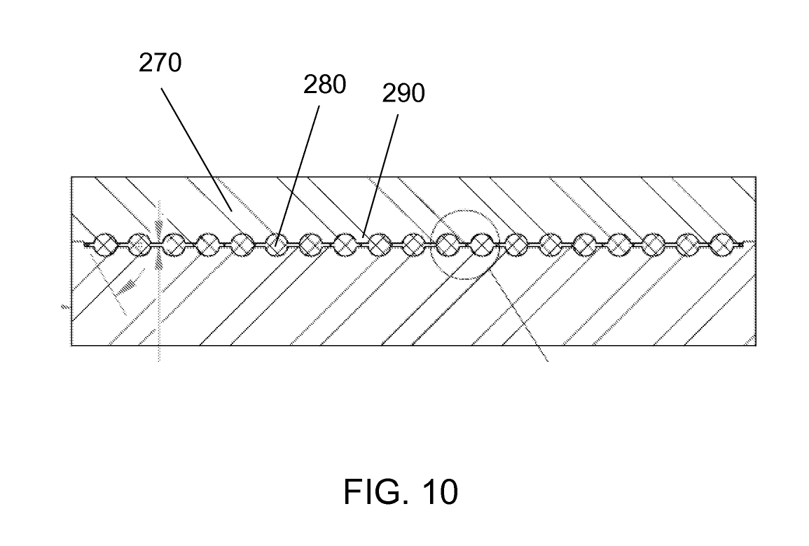

Both lower body 220 and upper body 210 have punch surfaces 230. Punch surfaces 230 on both upper body 210 and lower body 220 are designed such that when the surfaces are compressed together, a microtablet array 270 is formed as exemplified in FIG. 10. Microtablet array 270 comprises multiple microtablets 280 having a shape that is approximately spheroidal. Multiple microtablets 280 are connected to each other by depression region 290.

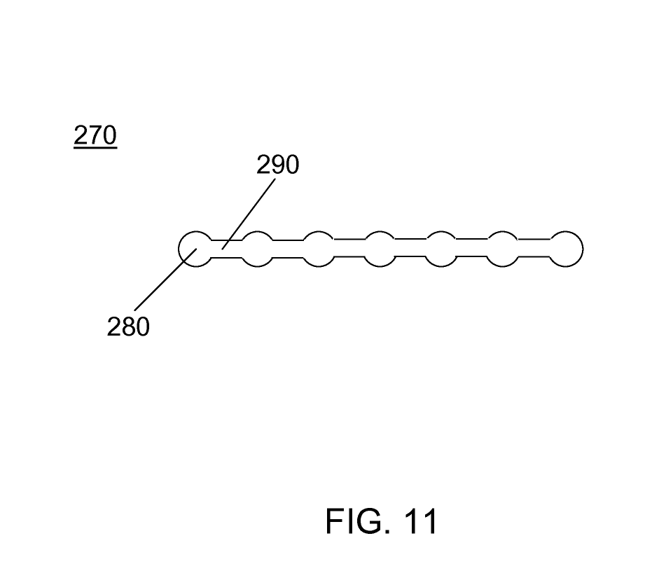

FIG. 11 shows an exemplary microtablet array 270 formed by assembly 200. Microtablet array 270 comprises multiple microtablets 280 connected by depression regions 290. Microtablet array 270 may have between about 2 and about 250 microtablets, preferably between about 4 and about 200 microtablets. The length and height of microtablet array 270 may vary depending upon the number and size of microtablets in the array. In certain embodiments, microtablet array 270 may have a length of between about 1 mm and about 25 mm. In other embodiments, microtablet array 270 may have a length of between about 10 mm and about 20 mm. In certain embodiments, microtablet array 270 may have a height of between about 0.25 mm and about 1 mm. Preferably, microtablet array 270 has a height of between about 0.4 mm and about 0.9 mm. After formation of microtablet array 270, individual microtablets 280 may be separated from microtablet array 270 by breaking the array at depression regions 290. Microtablets 280 may then be polished to remove depression region fragments from microtablet 280 and to form microtablet 280 to a substantially spherical shape. Microtablet 280 may be formed in a variety of sizes. In certain embodiments, microtablet 280 has a diameter of between about 0.25 mm and about 1 mm. Preferably, microtablet 280 has a diameter of between about 0.4 mm and about 0.9 mm. Preferably, microtablet 280 is substantially spheroidal, but microtablet 280 may have a variety of shapes depending upon the needs of the user.

The present invention is further described by reference to the following Examples, in which all parts and percentages are by weight, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

Example 1

### Preparation of Pressed Microtablets

Pressed microtablets containing a homogeneous dispersion of naltrexone were prepared by wet granulation as described below. The ingredients include naltrexone, 90.0g, lactose, hydrous (Sheffield) 450.0, polyvinyl alcohol (Colorcon) 60.0 g, purified water 35.0 g, fumed silica (Cabot) 6.0 g and magnesium stearate (Mallinckrodt), 6.0 g. All excipient materials were screened through a 20 mesh prior to use. The naltrexone was screened through a 40 mesh.

The naltrexone,lactose and polyvinyl alcohol were charged into a 5 qt planetary mixer and mixed dry for 5 minutes. Purified water was added over a period of 5 minutes with mixing. After the purified water was added, the mixing was continued for a period of 20 minutes. The granulation was discharged onto a paper lined tray and placed in an oven at 50° C. for a period of 16 hours. After 16 hours the dried granules were removed from the oven and passed through a 30 mesh US standard screen. The screened granules, silica and magnesium stearate were charged into a blender and blended for a period of 5 minutes. The blended materials were then charged into the hopper of a Picolla tablet press fitted with tooling designed to produce compressed microtablets having a diameter of 0.87 mm as exemplified in FIGS. 1-6 and comprised a lower punch body, upper punch body, lower pin holder, upper pin holder, eighty-seven upper pins and eighty-seven lower pins and a die. The microtablets were compressed at a pressure of 10 to 500 MPa.

### Example 2

### Preparation of Pressed Microtablets (Tooling Flipped On Its Side)

Pressed microtablets containing a homogeneous dispersion of naltrexone are prepared by direct compression as described below. The ingredients include naltrexone, 444.44 g, lactose, hydrous (Foremost) 545.56 and magnesium stearate (Mallinckrodt), 10 g. All excipient materials are screened through a 20 mesh prior to use. The naltrexone is screened through a 40 mesh.

The naltrexone and lactose are charged into a 16 qt V-type blender and blended for a period of 15 minutes. The magnesium stearate is added to the mixture and blended for an additional 5 minutes. The dry blended materials are then charged into the hopper of a tablet press as exemplified in FIGS. 7-11. A microtablet array is formed and compressed to a breaking force (hardness) of approximately 5-10 Newtons. Individual microtablets are removed from the array by fracturing the array along the depression. The individual microtablets are polished and the resultant microtablets are substantially spheroidal with a length of 0.85 mm and a mean diameter of 0.85 mm.

What is claimed:

1. An oral dosage form comprising a compressed microtablet, wherein said microtablet has a major dimension that is between about 0.25 mm and about 1 0 mm and comprises at least about 0.01 weight percent of at least one pharmaceutically active agent that is distributed substantially throughout said microtablet.

2. The oral dosage form of claim 1, wherein the pharmaceutically active agent is an analgesic, anti-inflammatory agent, anti helminthic, anti-arrhythmic agent, anti-bacterial agent, anti-viral agent, anti-coagulant, anti-depressant, anti-diabetic, anti-epileptic, anti-fungal agent, anti-gout agent, anti-hypertensive agent, anti-malarial, anti-migraine agent, anti-muscarinic agent, anti-neoplastic agent, erectile dysfunction improvement agent, immunosuppresant, anti-protozoal agent, anti-thyroid agent, anxiolytic agent, sedative, hypnotic, neuroleptic, beta-blocker, cardiac inotropic agent, corticosteroid, diuretic, anti-parkinsonian agent, gastrointestinal agent, histamine receptor antagonist, keratolytic, lipid regulating agent, anti-anginal agent, COX-2 inhibitor, leukotriene inhibitor, macrolide, muscle relaxant, anti-osteoporosis agent, anti-obesity agent, cognition enhancer, anti-urinary incontinence agent, nutritional oil, anti-benign prostate hypertrophy agent, essential fatty acid, non-essential fatty acid, or mixture thereof.

3. The oral dosage form of claim 1, wherein the compressed microtablet has a major dimension that is between about 0.4 mm and about 0.9 mm.

4. The oral dosage from of claim 1, wherein the compressed microtablet has a major dimension that is between about 0.5 mm and about 0.8 mm.

5. The oral dosage form of claim 1, wherein the compressed microtablet has an aspect ratio of between about 1:4 and about 1:1.

6. The oral dosage form of claim 1, further comprising at least one excipient.

7. The oral dosage form of claim 6, wherein the at least one excipient comprises a compression aid, a binding agent, a glidant, a disintegrant, a lubricant, or a combination thereof.

8. The oral dosage form of claim 7, wherein the compression aid comprises microcrystalline cellulose, lactose, dicalcium phosphate, sucrose, stearic acid, polyethylene glycol, waxes or a combination thereof.

9. The oral dosage form of claim 6, wherein the binding agent comprises hypromellose, hydroxyethyl cellulose, hydroxypropyl cellulose, methyl cellulose, cellulose ethers, cellulose esters, ethyl cellulose, cellulose acetate phthalate, hypromellose acetate phthalate, polyvinyl acetate phthalate, polyvinyl pyrrolidone, polyvinyl alcohol, copovidone, a carbomer, amino methylacrylate copolymer, methacrylic acid copolymers, acrylic polymers or a combination thereof.

10. The oral dosage form of claim 6, wherein the glidant comprises talc, silicon dioxide, metallic silicates, or a combination thereof.

11. The oral dosage form of claim 6, wherein the lubricant comprises calcium strearate, magnesium stearate, zinc stearate, stearic acid, talc, hydrogenated vegetable oil, glyceryl monostearate or a combination thereof.

12. The oral dosage form of claim 1, further comprising a coating over the microtablet comprising a water soluble polymer, a water retardant polymer, a pH dependant enteric polymer, or a combination thereof

13. The oral dosage from of claim 12, wherein the coating is a water soluble polymer.

14. The oral dosage form of claim 13, wherein the water soluble polymer comprises at least one alkyl cellulose polymer including cellulose, hydroxyethyl cellulose, hydroxypropyl cellulose, methyl cellulose, acrylic polymer, vinyl polymer, or a combination thereof.

15. The oral dosage form of claim 12, wherein the coating is a water retardant polymer.

16. The oral dosage form of claim 15, wherein the water-retardant polymer comprises at least one alkyl cellulose polymer, acrylic polymer, acrylic acid polymer, acrylic acid copolymer, methacrylic acid polymer, methacrylic acid copolymer, shellac, zein, hydrogenated vegetable oil, or a combination thereof.

17. The oral dosage form of claim 14, wherein the alkyl cellulose polymer comprises ethylcellulose or an aqueous dispersion of ethylcellulose.

18. The oral dosage form of claim 14, wherein the acrylic polymer comprises methylmethacrylate and ethylacrylate copolymer, ammonio methacrylate copolymer, or a combination thereof.

19. The oral dosage form of claim 16, wherein the methylmethacrylate and ethylacrylate copolymer is Eudragit NE 30D or Eudragit NE 40D.

20. The oral dosage form of claim 12, wherein the coating is a pH dependent enteric polymer.

21. The oral dosage form of claim 20, wherein the pH dependent enteric polymer comprises cellulose acetate phthalate, methacrylic acid copolymer, methacrylic acid copolymer dispersion, methacrylic acid copolymer, polyvinyl acetate phthalate, hydroxymethylcellulose phthalate, or a combination thereof.

22. The oral dosage form of claim 12, further comprising a lubricant added to the coating.

23. The oral dosage form of claim 22, wherein the lubricant comprises calcium strearate, magnesium stearate, zinc stearate, stearic acid, talc, glyceryl monostearate or a combination thereof.

24. The oral dosage form of claim 12, further comprising an enteric layer, a sealing layer, or a combination thereof coated on the coating.

25. A method comprising:
compressing a composition comprising at least one pharmaceutically active agent into a microtablet array having a height of between about 0.25 mm and about 1 mm and a width of between about 0.5 and about 25 mm;
breaking the compressed microtablet array perpendicular to its width to form multiple microtablets; and
polishing the microtablets to form at least two relatively spherical microtablets having a major dimension that is between about 0.25 and about 1.0 mm.

26. The method of claim 25, wherein the microtablet array is broken into at least two microtablets.

27. The method of claim 25, wherein the microtablets are formed at a rate that is greater than about 5,000 microtablets per minute.

28. A method comprising:
compressing a composition comprising at least one pharmaceutically active agent into a microtablet having a major diameter of between about 0.25 mm and about 1.0 mm.

29. The method of claim 28, wherein the microtablet is substantially spherical.

30. The method of claim 28, wherein the microtablets are formed at a rate that is greater than about 5,000 microtablets per minute.
